(19) United States
(12) Patent Application Publication
(10) Pub. No.: US 2013/0130933 A1
(43) Pub. Date: May 23, 2013

(54) BIOMARKER SIGNATURES FOR WELLNESS TESTING

(71) Applicants: John T McDevitt, Houston, TX (US); Pierre N. Floriano, Houston, TX (US); Nicolaos Christodoullides, Houston, TX (US); Glennon Simmons, Houston, TX (US)

(72) Inventors: John T McDevitt, Houston, TX (US); Pierre N. Floriano, Houston, TX (US); Nicolaos Christodoullides, Houston, TX (US); Glennon Simmons, Houston, TX (US)

(73) Assignee: WILLIAM MARSH RICE UNIVERSITY, Houston, TX (US)

(21) Appl. No.: 13/813,613

(22) PCT Filed: Aug. 11, 2011
(86) PCT No.: PCT/US11/47431

§ 371 Date: Jan. 31, 2013

Related U.S. Application Data
(60) Provisional application No. 61/372,961, filed on Aug. 12, 2010.
(60) Provisional application No. 61/406,224, filed on Oct. 25, 2010.

(51) Int. Cl. G01N 33/68; G06F 19/00

(52) U.S. Cl. CPC G01N 33/6893; G06F 19/34
USPC 506/9; 506/18; 702/19

(57) ABSTRACT

This invention generally relates to the use of devices to measure and assess the level of biomarkers that are indicative of the general wellness of an individual and methods of correlating such information into a wellness index.

### CROSS-REFERENCE TO RELATED APPLICATIONS

This application claims priority to 61/372,961, filed Aug. 12, 2010, and 61/406,224, filed Oct. 25, 2010, both of which are incorporated herein by reference in their entirety.

### FEDERALLY SPONSORED RESEARCH STATEMENT

This invention was made with government support under Grant No: 3U01DE017793-05S1 awarded by the National Institute of Dental and Cranofacial Research of the National Institutes of Health. The government has certain rights in the invention.

This invention was made with government support under Grant No: 5U01DE017793-07 awarded by the National Institutes of Health. The government has certain rights in the invention.

### FIELD OF THE INVENTION

This invention relates generally to the fields of medicine, physiology, diagnostics, and biochemistry. In certain embodiments, the invention relates to assessment of biomarkers indicative of the general health and wellness of an individual.

### BACKGROUND OF THE INVENTION

The traditional practice of the modern medicine and healthcare is reactive rather than preventive—curing diseases after their occurrence instead of promoting general health and preventing disease from occurring in the first place. With the rising cost of health care, there is an increasing need to evaluate an individual's general health or wellness condition so as to predict and hopefully prevent the onset of disease. A quantitative measurement of the individual's health would therefore be particularly useful to physicians and other healthcare providers. Moreover, a quantitative measurement can convey to a more precise assessment of an individual's health condition, possibly alerting that individual to change certain lifestyle or environmental variables so as to improve his or her health.

Despite the benefits that a quantitative measurement of an individual's health might provide, conventional measurements tend to be limited to different, unrelated scores pertaining to distinct aspects of an individual's body and biological system. Accordingly, it is difficult to provide a total wellness assessment of an individual.

Additionally, patient access, convenience and cost are issues that prevent the widespread use of wellness testing. The invasive and bio-hazardous nature of venous puncture-derived blood and delays associated with laboratory processing are not amenable to retail site test application and contribute significantly to cost. Although some point of care diagnostics are now available on the market, they tend to be directed to single tests, such as a test for urinary infection or for HIV infection, and thus are of very limited applicability. Likewise, test sensitivity and analyte range of the typical point of care diagnostic can also limit the usefulness of such tests.

Thus, what is needed in the art is a point of care diagnostic that measures many different analytes, providing an overall wellness index, and which has low cost, great sensitivity and range, as well as high reproducibility. Ideally the point of care diagnostic would employ saliva, a sample that is easily collected in any point of care setting, including retail outlets.

### SUMMARY OF THE INVENTION

Abbreviations

| | |
|---|---|
| Ab | Antibody |
| BM | Biomarker |
| BM_{L} | Biomarker level |
| BM_{T} | Biomarker Threshold Level (BM can be replaced with any |
| | biomarker abbreviation, thus it can be CRP_{T} etc.) |
| ΔBM | BM_{L} − BM_{T} |
| CHD | Coronary Heart Disease |
| CRP | C-reactive protein |
| DSA | double sided adhesive |
| DW | disease-specific weighing factor |
| hs-CRP | High sensitivity C-Reactive Protein |
| ID_{Disease} | index of death for a particular disease |
| ID_{Total} | Index for Total Risk for Death sum of all the ID_{Disease} for all diseases |
| LongWell Index | longevity/wellness index = (100 − ID_{Total}) expressed in % total life |
| | expectancy |
| PMMA | Poly(methyl methacrylate) |
| SS | stainless steel |
| SSA | single sided adhesive |
| THC | Δ9 — tetrahydrocannabinol |
| W_{DISEASE-BM} | Weighing factor for a particular marker for a particular disease |
| ΔDISEASE_{BM} | Δ_{BM} for a particular disease weighted by W_{DISEASE-BM} |
| ΔDISEASE | Sum of the ΔDISEASE_{BM} for all BMs considered |
| DNA | Deoxyribonucleic acid |
| RNA | Ribonucleic acid |
| LOC | lab on a chip |
| ABS | Acrylonitrile butadiene styrene |
| HIV | Human Immunodeficiency Virus |

The point of care diagnostic tests described here are novel in that they are designed to be performed on a programmable portable device that offers a fully quantitative and highly precise result. The device itself has a small footprint, and preferably uses disposable microfluidics such as are commonly found on lab-on-chip devices. Further, the tests demonstrate ultra low limits of detection and wide assay ranges that, in some cases, span up to six orders of drug concentration. To our knowledge there is no competitive technology available in this area.

Although our tests are preferably performed on a lab-on-chip platform, the biomarker panels are highly customizable, cost-effective, and adaptable to a number of platforms. Additionally, one or more biomarkers can be added or withdrawn from the panel according to the need of a particular industry or for a particular purpose, such as drug testing, cancer treatment, or inflammation monitoring.

Various diseases may affect the general well being of an individual. On the top of the list are the cardiovascular diseases, cancer, and diabetes. Biomarkers for high levels of inflammation may also be included in the current invention. High levels of inflammation are indicative of allergic reactions, acute conditions, trauma, and general infections, and also contribute to cardiovascular and other diseases. Allergy markers can also be included.

Besides the life-affecting diseases discussed above, certain behaviors may also affect the general health condition of an individual, including, most prominently, smoking and substance abuse. Cotinine or CRP can be used as a biomarker for detecting smoking behavior, while the popular biomarkers for detecting drug-abuse include, but are not limited to, amphetamine, cocaine, diazepam, and Δ9-tetrahydrocannabinol (THC), and many other drugs can be added to or substitute in this panel.

Other health-affecting behaviors and/or biomarkers can also be used in the method described in the current invention, especially common infectious diseases such as HIV, pneumococcus, and the like.

After the data on biomarker levels are collected, a scoring index can be generated, based on which the subject's general health condition can be determined. One way of generating or classifying biological data into an index system is described in US20080300798, the entire content of which is incorporated by reference into the current application. However, this application describes only a very simple index that doesn't incorporate diverse markers from diverse disease states or risk behaviors.

This new method for the assessment of Illness/Wellness and risk for death is based on the use of biomarkers (BM) associated with the development and/or diagnosis of high impact (high morbidity/mortality) disease, such as cardiovascular disease, cancer, diabetes, auto-immune disease, and of risky lifestyle behavior that contributes to disease and death, such as smoking, alcohol and use drugs of abuse, that are measurable in bodily fluids, such as serum, saliva and urine.

Examples of some of the BMs for the following areas of disease (and leading causes of death) as well as of RFs for risky lifestyle behavior, targeted by this invention are provided below:

TABLE 1
Preferred Biomarkers for use in the invention

Cardiovascular (cardiac, stroke etc.) BMs:
C-reactive protein (CRP)
soluble CD40 ligand (sCD40L)
monocyte chemoattractant protein-1 (MCP-1)
myeloperoxidase (MPO)
interleukin-1beta (IL-1β)
IL-6
tumor necrosis factor-alpha (TNF-α)
Homocysteine
phospholipase A2 (PLA2)
Cardiac troponin I (cTnI)
Cardiac troponin T (cTnT)
Myoglobin
CK-MB
d-dimer
Apolipoprotein A1 (apoA1)
Apolipoprotein B (apoB)
Brain natriuretic peptide (BNP) and N-Terminal proBNP (NT-ProBNP)
Lipoprotein A (LpA)
Cancer BMs:
Carcinoembryogenic antigen (CEA)
Cancer antigen 125 (CA125)
Her2-neu
CA15-3
Diabetes BMs:
Glycosylated hemoglobin (HbA1c)
Glycated albumin
Human Serum Albumin (HSA)
Smoking RFs:
Cotinine
Drugs of abuse RFs:
Cocaine
Diazepam
Δ9—tetrahydrocannabinol (THC)
Amphetamine

This new method incorporates information on biomarker(s) (BM) and their levels (BM_{L}) in the biological fluid (saliva, serum and urine) of an individual to derive: 1) The individual's BM-based index for risk of developing or having a disease; 2) The individual's BM-based index for risk for death; and/or 3) The individual's BM-based index of wellness and longevity.

The index aims to take into account what is known (published) about the association of biomarkers and risk factors with specific diseases, and weigh the measurements of these biomarkers that span numerous diseases for one particular patient using published statistics associated with the mortality and morbidity of the disease, in order to produce a number or index of wellness and longevity. In order to compile the index, levels of biomarkers that have precedent for a particular disease are measured for an individual, compared against established norms (normal physiological range), each of them being discounted by a weighting factor that corresponds to the association of a particular biomarker with a disease or condition (published, or experimental value). All the various weighted disease markers are summed up to generate a risk value for a person to have a particular disease based on that person's level of given biomarkers. This risk value is then discounted with a weight corresponding to the mortality of the disease and summed up across all the disease risks for which this person is being evaluated to create the index.

Thus, according to one aspect of the current invention, there is provided with a method for assessing the health and wellness status of a subject comprising the steps of: (a) measuring the level of a plurality of biomarkers in a sample from a subject, preferably from at least a cancer, diabetes, and cardiovascular biomarkers, (b) evaluating each biomarker level in said sample; and (c) determining a value representative of the health and wellness status or “index” based on an evaluation and transformation of biomarker levels. Traditional biomarker evaluation can be advantageously combined with other factors such as being overweight, high body mass index or high abdominal fat, smoking, drug and alcohol use or abuse, high stress levels, age, gender, ethnicity and so on.

Immediate applications of the current invention include wellness monitoring for large populations to establish baselines such as in the military or schools, underserved communities, and enabling local, regional, or global health surveillance. Another field of use could be monitoring of populations at risk because of potential harmful exposure to toxins in their work or monitoring of risky health behavior for both legal and counseling aspects. Ultimately, wellness testing can become as commonplace as blood pressure testing now offered in chain drugstores, and thus be generally available to the population.

According to preferred embodiments, the biomarkers are for detecting cardiovascular disease, cancer, diabetes, inflammation, as well as optionally smoking, drug-abuse, infectious disease, or combinations thereof. Preferably, the biomarkers are selected from the group listed in Table 1 above.

In preferred embodiments, the assays are protein and antibody based, and one or more antibodies are conjugated to fluorescent labels, but any target and detection method can be used. Thus, any “target-detector binding pairs” can be used, including but not limited to DNA-DNA, DNA-RNA, glycoprotein-leptin, enzyme-substrate, receptor-ligand, and other target detection pairs can be employed, as well as any labels or detection methods, many of which are known in the art.

In some embodiments, the sample tested is body fluids collected via non-invasive ways, such as saliva or other readily available fluid, but various samples can be used in the method of the current invention. Examples include, but are not limited to, tears, nipple aspirate, serum, blood, cerebrospinal fluid, saliva or other oral fluid specimen, urine and biopsy samples, but the preferred sample is saliva due to its ease of collection in a point of care environment.

In particular, the invention include a non-invasive, pain-free assessment/classification of general health condition using saliva, which, when used in conjunction with a point of care device, introduces the possibility of a home- or retail-based health assessment test. The method can be completed at the point-of-care, enabling more rapid and effective assessment of health condition and, hence, the reduction of health care costs. The method can also be used to gauge the efficacy of treatment and guide future interventions or therapy.

The current invention is particularly useful to assessing the general health condition of a human subject. However, the current invention is equally applicable to other subjects such as animals, including livestock, companion and exotic animals, fish, poultry, and the like.

The output from a detection device can be processed, stored, and further analyzed or assayed using a bio-informatics or a computer system. A bio-informatics system can include one or more of the following: a computer; a plurality of computers connected to a network; a signal processing tool(s); and an algorithm. In some embodiments, the data is sent to a central databank via the web, and is accessible to the patient and/or healthcare provider. In some embodiments, as wellness report is generated providing individualized data as well as an overall wellness index and optionally graphically representing the subjects overall wellness.

In other embodiments of the invention, a microelectronic device, such as lab-on-a-chip (LOC), is used to conduct the measurement. As a clinical research tool, the LOC device offers the ability to perform multiplex assays in small sample volumes. Additionally, the versatility of this system and its demonstrated enhanced sensitivity makes it more a more sensitive biomarker validation tool, while at the same time amenable to applications involving a variety of bodily fluids, such as saliva or other oral fluid specimen, in which the analyte concentration may be extremely low. For example, salivary biomarkers that were previously undetectable by standard methods, may now be targeted with the LOC device to assess systemic disease in a non-invasive fashion. Examples of such LOC are set forth in Goodey et al., J. Amer. Chem. Soc., 123(11):2559-2570, 2001, and Christodoulides et al., Lab. Chip, 5(3):261-9, 2005b, the entire contents of which are incorporated by reference into this application.

In certain embodiments, the invention is directed to a novel LOC platform or disposable cassette, wherein the testing sites comprise a porous agarose surface that is conjugated to either target or anti-target antibody, and thus serves in competitive or sandwich immunoassays. Generally, the LOC thus comprises a membrane or other porous surface through which fluid can be flowed, and agarose pancakes or disks are spotted thereon, each pancake dedicated usually to a specific target.

The agarose is conjugated to one member of a target-detector binding pair, as indicated by the assay needs. The conjugation can occur before of after spotting, but it preferably done before spotting to ensure consistent dispersal of the reagents throughout the disk. The use of a flat agarose disk eliminates the variability that is introduced into the assay with the use of beads of varying sizes, and also eliminates the variability that exists across the diameter of a single bead caused by surface effects and differential penetration of reagents into the bead. By “flat” herein what is meant is that the pad if at least five times wider in both dimensions than its height, and preferably is about 10× wider than high.

In some embodiments, where a thin porous membrane is used, it can be further supported on an additional support that is glass, plastic, ceramic or a semiconductor chip substrate. The substrate preferably contains channels and other microfluidics such that fluid can be forced to pass through the agarose and porous membrane, and collected and disposed of Preferably, the entire assembly is a disposable cassette, that fits into as portable LOC device that provides the electronics, pumps, controls and other systems needed to operate the assay and display or print out assay results. However, where the porous substrate is not membrane based, but is instead a porous glass or ceramic fit, this secondary support is optional.

In some embodiments, the testing sites are arranged on disposable cassettes, such as glass or plastic slides onto which fluid channels can be placed or etched or engraved. A porous surface, such as a membrane or frit, overlays the microfluidics, and the agarose disks are spotted thereon. Disk (circular) shapes are preferred as easy to achieve with simple fluid spotting techniques, but other shapes are possible depending on the method of applying the agarose.

The porous substrate can be any membrane, such as nitrocellulose membrane, or Poly(methyl methacrylate) PMMA can be a more substantive support, such as porous glass, ceramic, plastic (delrin, PMMA, Acrylonitrile butadiene styrene, i.e. ABS), or metallic (e.g., stainless steel) frit.

The agarose can be plain agarose, or any of the agarose derivatives such as cross-linked agarose, sepharose, or any agarose derivatives that can be used for affinity chromatography. The disk is preferably about 10-50 um thick and 50-200 um in width, but larger or smaller sizes are also possible, depending on sample size, specificity of the reagents, and the sensitivity of the instrumentation.

Although exemplified herein with protein antibody target detection pairs, the agarose disc platform can be used with other target-detector binding pairs such as described above.

### BRIEF DESCRIPTION OF THE DRAWINGS

FIG. 1. Dose response curve for the THC assay. Since the assay was a competitive assay, signal declines with increasing THC in the sample.

FIG. 2. Dose response curve for the cocaine assay. Since the assay was a competitive assay, signal declines with increasing drug in the sample.

FIG. 3. Dose response curve for the amphetamine assay. Since the assay was a competitive assay, signal declines with increasing drug in the sample.

FIG. 4. Dose response curve for the diazepam assay. Since the assay was a competitive assay, signal declines with increasing drug in the sample.

FIG. 5. Dose response curve for the albumin assay. Since the assay was a sandwich assay, signal increases with increasing target in the sample.

FIG. 6. Dose response curve for the troponin assay. Since the assay was a sandwich assay, signal increases with increasing target in the sample.

FIG. 7A. Dose response curve for NT proBNP assay. Since the assay was a sandwich assay, signal increases with increasing target in the sample. FIG. 7B. Dose response curve for BNP assay.

FIG. 8: Cross section of flat agarose pad cassette.

FIG. 9. Top view of a single plastic holder (black) with circular hole into which gel (now shown) is placed and backed by membrane (dotted) is shown on the right. These individual gels pads can be grouped as desired, including in a circular format (middle) or clusters specific to each analyte can be grouped (right).

FIG. 10. One assay format showing gels can also be made into macroscopic shapes such as rectangles, squares, crosses, that can be visualized through the use of a colorimetric assay. Shown here are a structure before an assay (left), a negative result in the shape of a minus sign (middle) where only the positive control lights up, and a positive result (right) where both the positive control and the sample show signal, thus printing a positive sign.

FIG. 11. Top view showing lateral flow across an agarose pad cassette.

FIG. 12. Top view of agarose filled channels.

FIG. 13A is Table 2A and FIG. 13B is Table 2B and are reagents for the drug assays tested herein.

### DESCRIPTION OF EMBODIMENTS OF THE INVENTION

The use of the word “a” or “an” when used in conjunction with the term “comprising” in the claims and the specification means “one or more than one.”

Throughout this application, the term “about” is used to indicate that a value includes the standard deviation of error for the device or method being employed to determine the value. Where no standard range of error in measurement is available or readily ascertainable, the term “about” means +/−10%.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The following description aims to provide more detailed description of the invention and to illustrate the general principles of the invention. It should not be taken in a limiting sense. The section titles and overall organization of this section are adopted for the convenience of description and are not intended to limit the present invention.

The current invention is generally related to a process for using biological samples, preferably noninvasive oral fluid samples, for the measurement of panels of biomarkers that yield critical information related to key health parameters. These biomarker signatures can be used in the assessment of the future risk of persons for suffering live threatening diseases in the areas of cardiac disease, cancer, and diabetes. The same testing can be used to assess the status of patients participating in risky behaviors such as in the use of drugs of abuse and smoking. The reliable test described herein can be used to assess the disease risk factors and can be in some cases to provide advanced treatment and adjust behavior before adverse health effects are manifested.

According to certain aspects of the current invention, the use of the biomarker fingerprints involves the following steps. First, a biomarker panel is constructed by selecting one or more biomarkers from a list of biomarkers that are indicative of certain diseases or health-affecting behaviors (see e.g., Table 1). Samples are collected, and the panel of biomarkers is then measured on a suitable instrumentation platform that is capable of high fidelity measurements of protein levels. The levels of the biomarkers are translated to a risk assessment score, as follows:

Longevity/Wellness Index (LWI) expressed in % total life expectancy is determined according to equation 1):

LWI=(100−ID_{Total}) 1)

wherein ID_{Total} is the overall total index of death and is determined by equation 2):

ID_{Total}=ID_{Disease#1}+ID_{Disease#}₂+ . . . +ID_{Disease#n} 2)

wherein n is the number of diseases considered and ID_{Disease} is the index for risk for death from that disease is determined by equation 3):

ID_{Disease}=ΔDISEASE×DW 3)

wherein ΔDISEASE is Σ ΔDISEASE_{BM#1} . . . ΔDISEASE_{BM#m}

wherein m is the number of biomarkers considered, and DW is a disease-specific weighing factor and ΔDISEASE_{BM} is determined by equation 4:

ΔDISEASE_{BM}=(BM_{L}−BM_{T})×W_{DISEASE-BM} 4)

wherein BM_{L} is the biomarker level in the patients and BM_{T} is the biomarker threshold level which is cut-off point established for the specific marker above or below which disease occurs, and wherein W_{DISEASE-BM} is a weighing factor based on the published increase in risk associated with a particular level of biomarker.

By “biomarker” in the above equations, we define such to include all traditional biological biomarkers as well as life style risk factors, age, gender, ethnicity, fat levels, etc. Thus, regardless of the type of marker, the levels thereof can be employed to generate a wellness index. Furthermore, although we discuss a wellness index herein, it is apparent that such is easily converted to a illness index or risk of death index, as these are interrelated.

Finally, the risk profile is presented to the patient and/or the health care provider in a manner whereby the future prognosis and possible interventions can be placed into context. This information may then be used to adjust treatment or behaviors on a time frame where major disease damage has not yet occurred.

In more detail, the invention uses nanoscale lab-on-chip (“LOC”) competitive immunoassay for target analyte (“target”), is described as follows:

An anti-target tracer antibody is mixed with the sample and delivered to the testing site, wherein agarose bead or flat disk was previously conjugated with BSA-target, as well as negative controls testing sites conjugated with BSA alone, as well as calibrator beads used as internal controls for the LOC system. In the absence of target in the sample, the anti-target tracer antibody will efficiently recognize the testing sites specifically coated with the drug and thus produce a fluorescent signal within and around the test site. In the presence of target, however, the signal on the test site loaded with BSA-target will be significantly reduced in a target-specific, dose dependent manner.

Alternatively, a sandwich immunoassay can be used, wherein one or more capture antibodies (anti-target antibody) is conjugated to the agarose bead or flat disk and is used to capture target from the sample. The antibody is then detected with a second anti-target antibody that binds to a different epitope of the target. Where an target is particularly difficult to detect or sensitivity is an issue, multiple capture antibodies can be employed and/or multiple levels of detection antibodies. The use of additional capture antibodies ensure that the target is captured, regardless of variation in confirmation or modification of the target, and the use of multiple detection antibodies allows amplification of the signal.

Whether employing beads or reaction wells or the novel agarose disk approach, the assay is preferably done in an array format with multiple testing sites for each sample, and multiple controls. The array approach of the lab-on-chip testing allows simultaneous assessment of testing sites loaded with varying amounts of target, thus improving the reliability of the assay. Further, the array format allows for the use of negative control beads (i.e. testing sites having with BSA alone) that provide an initial understanding of the specificity of the antigen-antibody reactions, such as of the binding of the tracer antibody on the bead or agarose disc testing sites or sensors.

### Example 1

### Bead Based Drug Assay

We have developed a four drug test to measure THC, cocaine, diazepam and amphetamine usage in patients. The test was a competitive immunoassay, as described generally above and was performed on porous agarose beads. The beads are sufficiently large (˜200 μm), that individual beads could be visualized and intensity of signal measured on a per bead basis.

We used 2%-6% cross-linked, glyoxylated agarose beads for the bead based assays. Agarose particles (6% crosslinked) used for the enzyme-based studies were purchased from XC Particle Corp. (Lowell, Mass.). The particles were glyoxal activated (20 moles of activation sites per milliliter) and were stored in sodium azide solution.

Agarose particle sizes ranged from 250 μm to 350 μm. Past research with these bead sensors consistently revealed that the precision of the assay was highly dependent on size homogeneity. Accordingly, an integral component of the bead production protocol included a sieving step where beads within a 280±10 μm diameter distribution were selected. However, some outlier beads occasionally appeared in the array. Also, since the agarose beads were porous, reagents not only attached to the surface, but also penetrated the bead interior and covalently bound thereto. However, bead penetration was a function of reagent exposure time and regent size, and thus contributed some degree of variability to the assay. Each of these variations can be compensated for in data analysis, however.

The reagents employed in these assays are shown in Table 2, appended behind the figures. Methodology was as indicated by the supplier, or as described in WO2004072613.

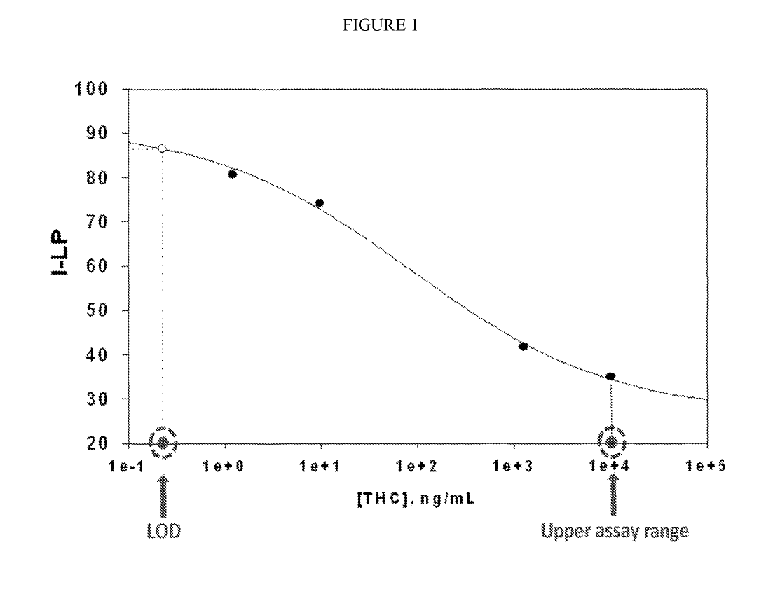

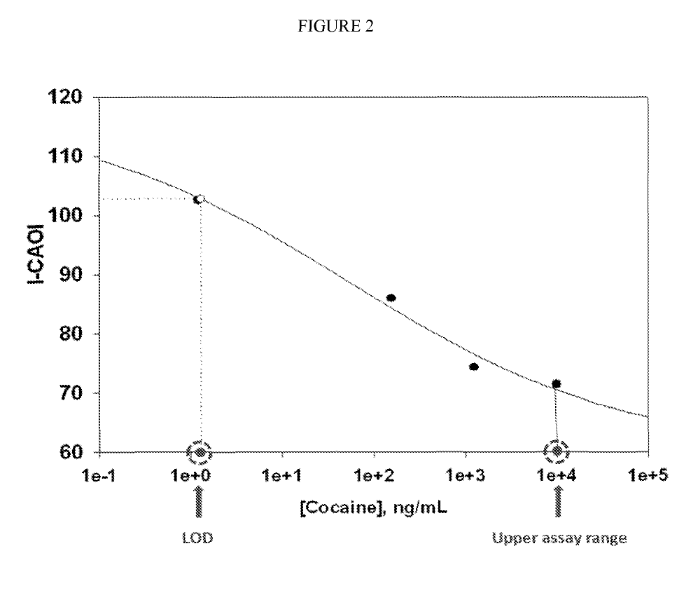

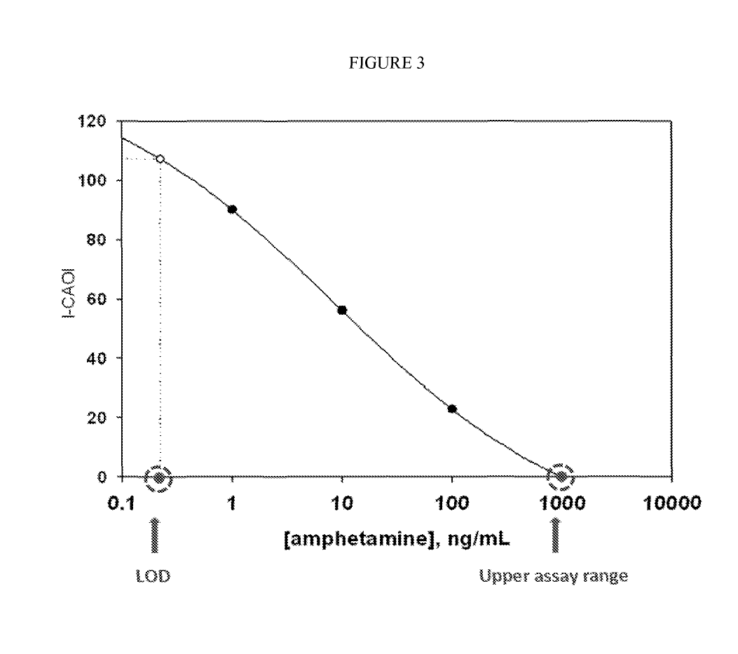

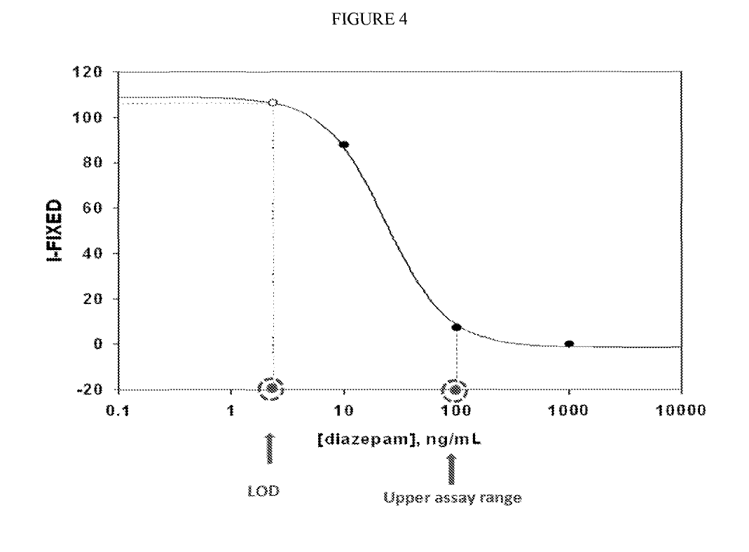

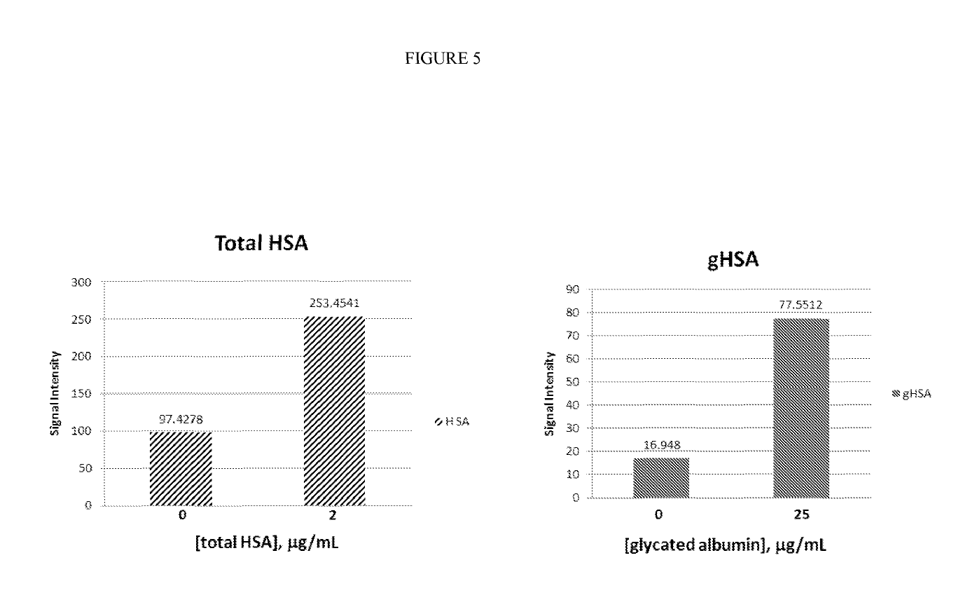

By using the above-described immuno-format and bead-loading strategy, we have demonstrated proof of concept of competitive immunoassays for all of the 4 drugs targeted in this program and the results are shown in Table 3, and the dose response curves are shown in FIG. 1-4.

The results prove that a high signal to noise result was achievable, whereby BSA-drug-specific beads provided a significantly higher signal than negative control (BSA-coated) beads in response to the tracer antibody. Further, for all 4 assays, we demonstrated efficient competition between analyte and tracer antibody, whereby in the presence of the specific analyte, the baseline signal on analyte-specific beads (in response to tracer antibody) was significantly reduced as compared to the zero antigen-control condition.

TABLE 3
TEST RESULTS FOR PANEL TESTING OF 4 DRUGS

| | Limit of detection (ng/mL) | Assay Range |
|---|---|---|
| Assay | 99.9% CI (3 × SD) | (ng/mL) |
| THC | 0.22 | 0.22-10,000 |
| Cocaine | 1.3 | 1.3-10,000 |
| Diazepam | 2.0 | 2.0-100 |
| Amphetamine | 0.22 | 0.22-1,000 |

### Example 2

### Bead Based Diabetes Assay

To assess diabetes risk, we measured glycated albumin and total human serum albumin in a sandwich assay, wherein an anti-HSA antibody that binds both albumin and glycated albumin was the capture antibody, conjugated to the beads as described above. Two additional antibodies, one specific for HSA and conjugated to alexafluor-488 (green fluor) and one specific to glycated albumin and conjugated to alexafluor-647 (red fluor) were used to then detect the captured analytes, respectively. A ratio of red/(green +red)*100 (i.e. glycated albumin/total albumin) was then used to measure the percentage of glycated albumin of the total albumin in the sample.

The following reagents were employed to accomplish this proof of principle test.

TABLE 4
ALBUMIN ASSAY REAGENTS

| | |
|---|---|
| Capturing Ab: | anti-HSA antibody; BioDesign Cat.# H45700M; |
| | 0.1 mg |
| Calibrator Beads: | Molecular probes Goat anti-mouse IgG-AF 488; |
| | 0.02 mg/ml |
| Antigen Standards: | gHSA; Exocell Cat.# NGA |
| | HSA; Sigma-Aldrich Cat.# A9511 |
| Detecting Ab: | anti-gHSA antibody; Exocell Cat.# A717 |
| | conjugated to AF-647 |
| | anti-HSA antibody; Biodesign Cat.# H86611M |
| | conjugated to AF-488 |
| Assay Conditions: | PBSA blocking: PumpA; 50% flow rate, 4 min. |
| | Ag + Ab priming: PumpB; 50% flow rate, 12 sec. |
| | Ag + Ab incubation: PumpB; 7% flow rate, 30 |
| | min. PBS wash: PumpB; 50% flow rate, 5 min. |

Bar graphs of proof of concept for the two assays are shown in FIG. 5.

### Example 3

### Bead Based Cardiovascular Assay

Troponin: Our initial biomarker studies demonstrated some potential of cTnI as a salivary biomarker of acute myocardial infarction (AMI), despite its low concentrations in this biological fluid. Evidence from our prior studies suggests the diagnostic capacity of a salivary cTnI test could be significantly improved with a much more sensitive LOC assay.

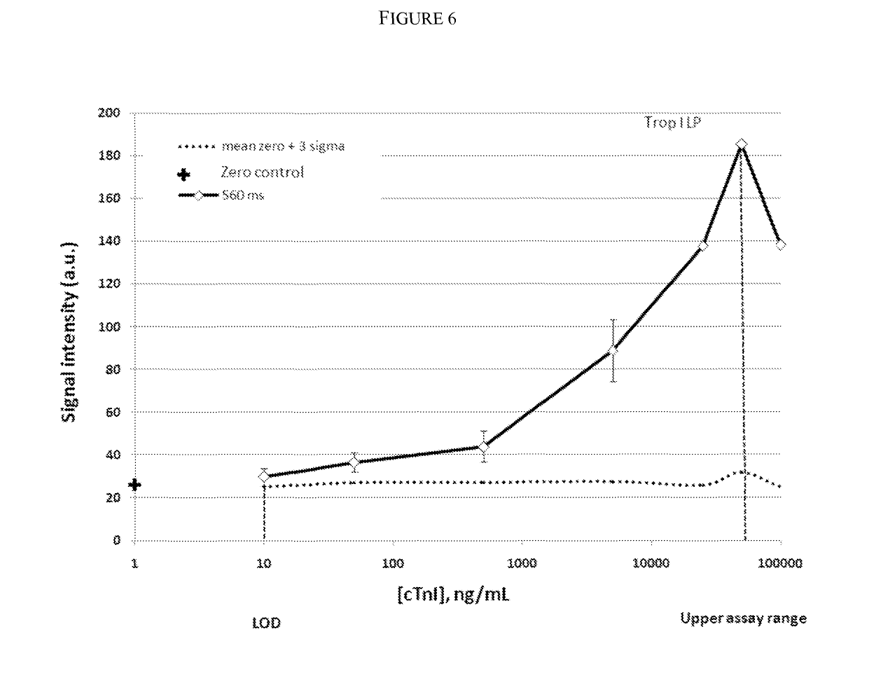

We therefore developed a very sensitive immunoassay in which cTnI capture on the beads was achieved through three distinct cTnI-specific capture antibody clones (thus capturing target regardless of tertiary or quaternary structure variations) and its detection was enhanced via a signal amplification scheme using two different (stacked) detection antibodies. Although still in the early stages of optimization, this cTnI assay has demonstrated in some experiments a limit of detection at 0.01 ng/mL. The dose response curve is shown in FIG. 6.

TABLE 5
REAGENTS USED FOR CTNI ASSAY

| Capturing Ab: | 3 monoclonal antibodies; IPOC [MA-1050, |
|---|---|
| | MA-1040, MA-1010] |
| Negative Control Beads: | TN F-α; 2mg |
| Calibrator Beads: | Molecular probes Goat anti-mouse IgG-AF 488 |
| | (From AMI Multiplex) 0.02 mg/m1 |
| Antigen Standard: | Fitzgerald cTnl standard; Cat. # 30R-AT034 |
| Detecting Ab: | Primary Ab: cTnl primary ab; Nanogen Cat# |
| | A1-PA-1010 |
| | 1:250 |
| | Secondary Ab: Goat anti rabbit IgG-AF 488; |
| | Invitrogen Cat# A11070 |
| | 1:500 |
| | 1:250 |

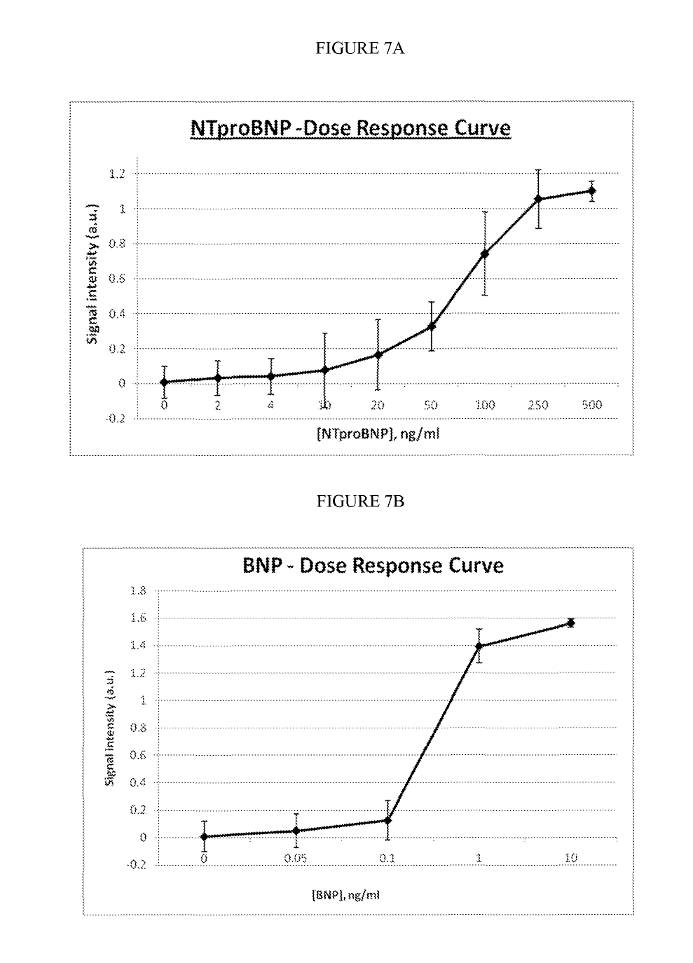

BNP and NT-proBNP: Inclusion of BNP or NT-proBNP would offer prognostic, along with diagnostic, information in chest pain patients with acute myocardial infarction. Dedicated efforts were thus employed for the development of a specific assay on the LOC platform as well as of NT-proBNP. An optimal antibody pair for the capture and detection of the BNP analyte using a “sandwich”-type, two site immunometric approach has been identified and applied to establish proof of principle for this important assay (see Table 6). Further, a calibration curve was developed for this new test demonstrating a limit of detection of 50 pg/mL and an upper limit of 10 ng/mL. The dose response curve is shown in FIG. 7A. BNP was also assayed (see table 7), and data shown in FIG. 7B.

TABLE 6
REAGENTS USED FOR NT PROBNP ASSAY

| | |
|---|---|
| Capturing Ab: | monoclonal antibody (1) Cat # H86511 m from |
| | Bio-design Intl (beads were conjugated with |
| | 4 ug antibody) |
| Negative Control Beads: | HSA-G5P01-04; 0.1 mg |
| Calibrator Beads: | Molecular probes Goat anti-mouse IgG-AF 488 |
| | (From AMI Multiplex) 0.02 mg/ml |
| Antigen Standard: | NTproBNP 1-76 ac. Cat# EK011-42 Phoenix |
| | Pharm. Inc. |
| Detecting Ab: | monoclonal antibody (2) Cat # H86214 m from |
| | Bio-design Intl conjugated to AlexaFluor 488 |

TABLE 7
REAGENTS USED FOR BNP ASSAY

| | |
|---|---|
| Capturing Ab: | monoclonal antibody (1) Cat #H 86507m from |
| | Bio-design Intl (beads were conjugated with |
| | 4ug antibody) |
| Negative Control Beads: | HSA-G5P01-04; 0.1mg |
| Calibrator Beads: | Molecular probes Goat anti-mouse IgG-AF 488 |
| | (From AMI Multiplex) 0.02 mg/ml |
| Antigen Standard: | BNP-32 (human), Cat#EK011-03 Phoenix |
| | Pharm. Inc. |
| Detecting Ab: | monoclonal antibody (2) Cat#H86245 m from |
| | Bio-design Intl conjugated to AlexaFluor 488 |

Furthermore, both assays for cTnI and NT-proBNP have been validated with testing of serum samples from AMI and Congestive Heart Failure (CHF) patients (data not shown).

### Example 4

### Agarose Disc Methodology

Because there were always limitations caused by the bead format as discussed in EXAMPLE 1, we have explored other formats to eliminate variation caused by bead size and differential bead penetration of reagents. We have a great deal of familiarity with the cross-linked, glyoxylated agarose beads, and thus decided to continue working with agarose and its derivatives, but sought to eliminate any geometric effects caused by the spherical bead by switching from a 3 dimensional format to a 2 dimensional format. Thus, the bead was replaced by a spot where agarose was dotted, and thus was generally pancake or disk shaped.

The first prototype gels pads were made by taping a form to glass slides, and dropping molten agarose (with various concentrations) into the mold with a pipette. When the gels hardened, the mold was removed leaving gel pads, in this case squares with rounded corners. 0.1%, 0.7%, 2% and 4% gel pads were made, but the 0.1% pads were discarded as too thin for use. However, thinner gels can be used where some crosslinker is added. Thinner gel pads can be obtained by placing cutouts, or gaskets of various materials (vinyl or various other plastics, Teflon, silicon, etc. . . .), size, diameters, and depth onto a glass slide. Similarly, the gel drops are deposited inside the gasket and a glass slide is applied to form flat pads. Alternatively, gel drops are formed if the pads are not covered while the gel is forming.

The pads were pulled off the slides, glyoxylated and conjugated with antibody or BSA according to known procedures (Goodey et al., J. Amer. Chem. Soc., 123(11):2559-2570, 2001). The antibodies and controls used in our proof of concept testing are listed below:

TABLE 8
REAGENTS FOR GEL PAD TESTS

| | |
|---|---|
| Positive control to | donkey anti-sheep (DαS) IgG labeled with |
| | Alexa ® |
| optimize optics | Fluor 488 (Invitrogen, Cat#A11015, Lot#687630) |
| Negative control | BSA |
| to optimize optics | |
| Capture antibody | Anti-CRP antibody (Fitzgerald, Cat#10 C33A) |
| Antigen (test sample) | CRP antigen (Fitzgerald Cat. #30-AC10) |
| Detection antibody | anti-CRP antibody (Fitzgerald Cat. #10-C33C). |
| | This was then conjugated to Alexa ® fluor 488 |
| | using the manufacturer's instructions |

The test pads were placed back onto glass slides, and tested for their ability to bind CRP and provide a detectable signal. The results, shown in Table 9 indicate that for at least this target and regents a better signal/noise ratio was achieved with the higher (4%) percentage agarose. However, it is easily possible to tune the agarose percentage and composition for every target binding pair combination. For example, agarose can be made superporous by using a surfactant in order to increase the size of the pores in the same way for the gel as is done for beads. (Gustaysson, P. E., et al., Superporous agarose beads as a hydrophobic interaction chromatography support, J. of Chromatog., A 830(2): 275-284 (1999); Gustaysson, P. E. and Larsson, P. O., Superporous agarose, a new material for chromatography, J. Chromatog. A 734(2): 231-240. (1996)).

TABLE 9
GEL PAD TEST RESULTS

| | CRP Assay Results | | | | | |
|---|---|---|---|---|---|---|
| | 0.7% | | 2% | | 4% | |
| | 0 ng | 100 ng | 0 ng | 100 ng | 0 ng | 100 ng |
| CRP | 34.212 | 69.534 | 46.998 | 115.397 | 24.872 | 128.499 |
| Neg | 46.243 | 40.66 | 49.797 | 45.404 | 35.225 | 45.06 |

Some modifications to the lab on chip cassette will be necessitated by the change to agarose pads so that the microfluidics are consistent therewith. However, this is easily accomplished by directing flow through the agarose pad. Flow can also be laminar, that is across the agarose pad, but it is expected that flow through the pad may produce a better sign/noise ratio, with the same flow rate, especially with agarose of higher concentration.

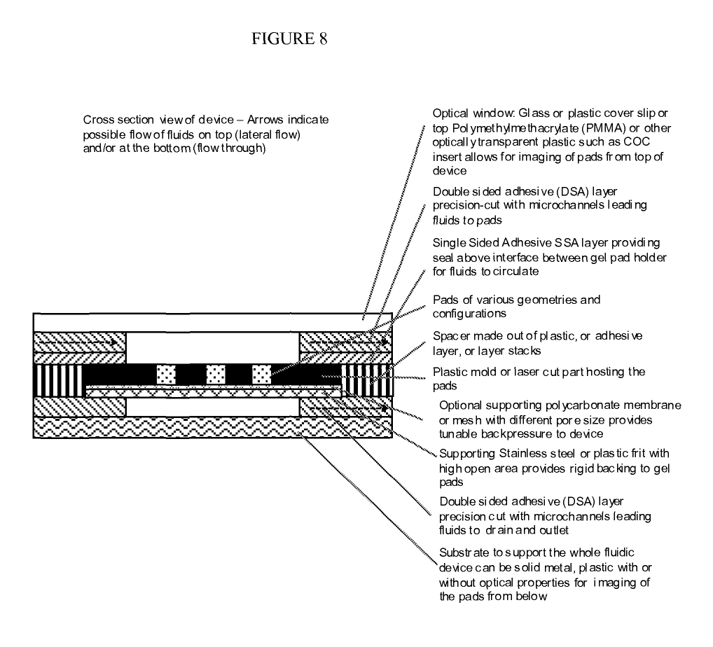

FIG. 8 shows a cross section of an LOC cassette with agarose disks. FIG. 8 simply shows the agarose pads on a membrane support with channels above and below same. Fluid enters the top left, travels through the pads and membrane, and exits right via the bottom channel.

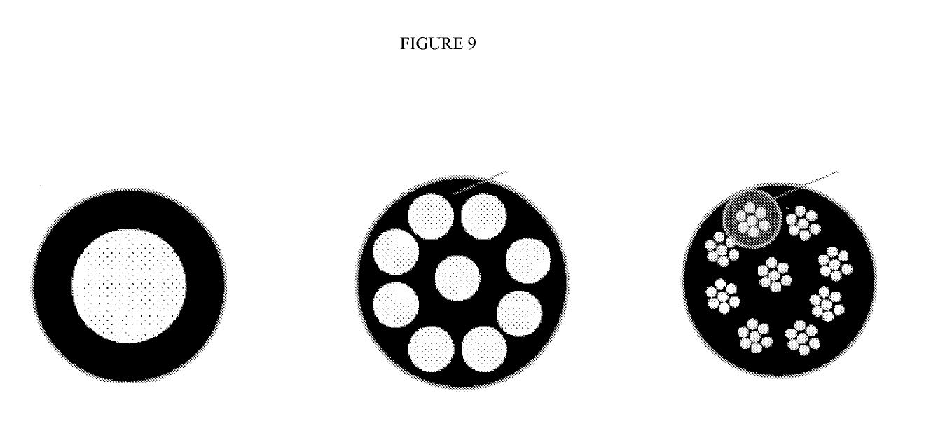

FIG. 9 shows a top view for another configuration of agarose pads, this time arranged in groups and the groups further arranged in circles.

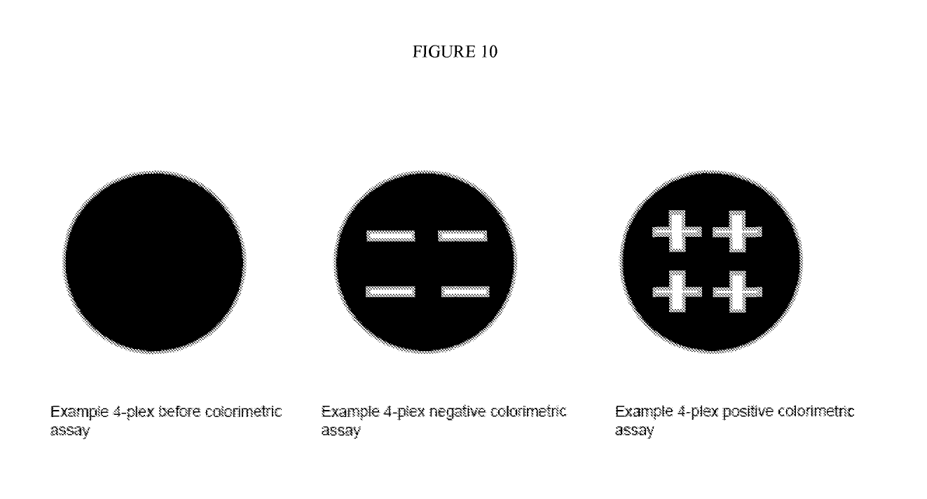

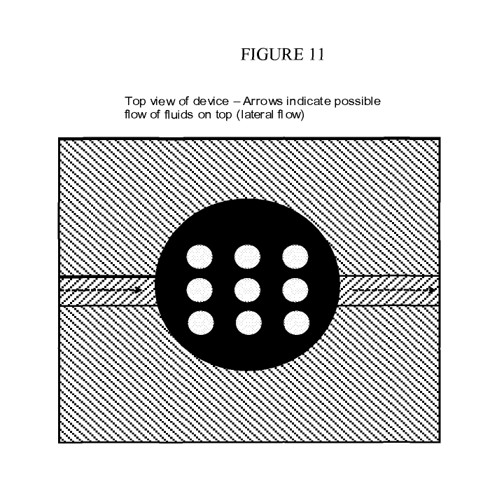

FIG. 10 shows an exemplary assay configuration indicating positive and negative results.

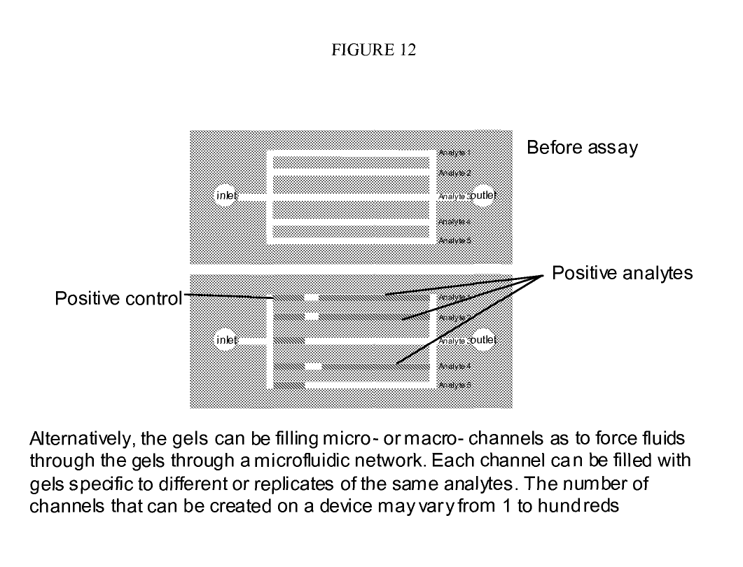

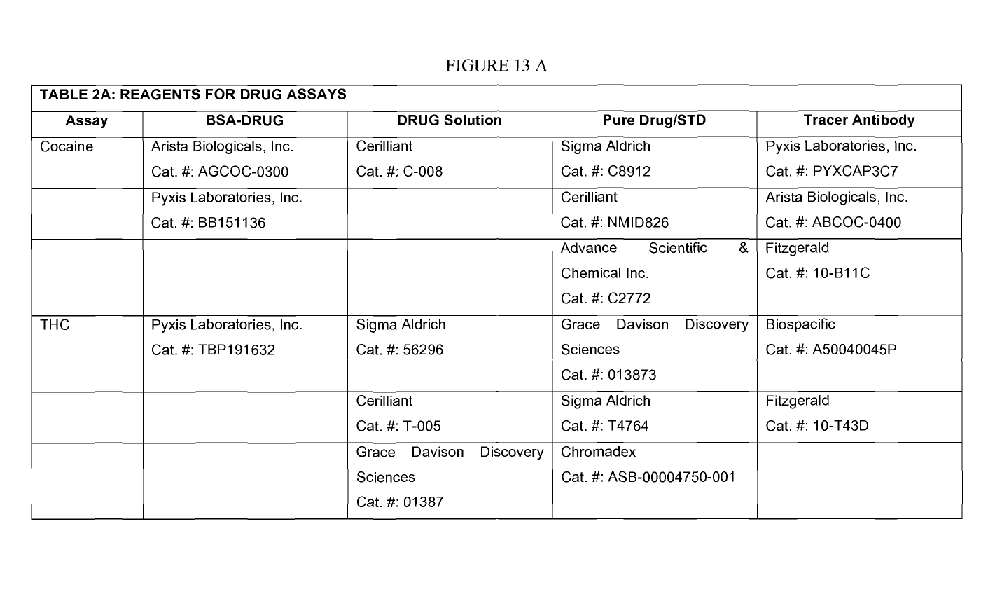

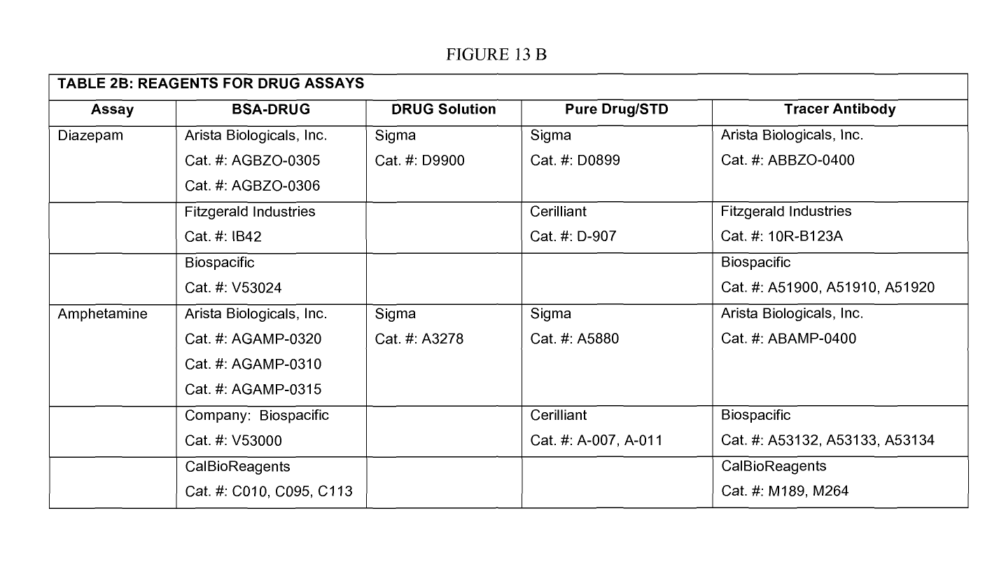

FIG. 11 shows lateral flow over the top of agarose pads, and FIG. 12 shows flow controlled by channel that are agarose filled. FIG. 11 is a top view of the device showing an inlet channel (left) and an outlet channel (right). In one mode of flow, the fluids circulate laterally from left to right in a flow over the agarose pads, while the channel at the bottom out of the drain is closed by a valve. In another mode, the channel leading out of the device (right) is closed by a valve, and the flow through mode of the fluids is from left inlet through the pads into the drain and out to waste. In another mode, both the outlet channel on top and at the bottom are opened and the pressure can be regulated by partially closing valves on top or at the bottom to generate pressure either favorable to lateral, or flow through mode. Fluidic lines and controls can be arranged to allow for fluids exiting the top and/or bottom outlets to be recirculated toward the inlet.

In FIG. 12, fluids are introduced in the inlet port (left) and forced through the gel-containing channels. A portion of each channel has been filled with gel that contains a positive control that serves as internal control that the sample has traveled through as well as the reagents are working properly. This portion should always develop a color, whether in the fluorescent or colorimetric mode. A second portion of the channels is filled with gel with a specific analyte. If color develops, it is because of the presence of the targeted analyte. Channels can be loaded with more than one slug, or section for a specific analyte. Fluids exit at the outlet (right). As in the device shown in FIGS. 8 and 11, fluidic lines and controls can be arranged to allow for fluids exiting the top and/or bottom outlets to be recirculated toward the inlet.

### Example 5

### Disease Index

The BM-based index for Risk incorporates a comparison of the level of each of the biomarkers (BM_{L}) with respect to either: a) reference range, b) discrimination limit or c) a disease threshold value.

As an example the BM_{L} is compared to the biomarker threshold level (BM_{T}) or cut-off point established for the specific disease. The BM_{T} is derived from the standard reference range of the specific biomarker's concentrations (for healthy individuals) and it is defined by the upper limit concentration from the healthy group above which risk for developing or having the disease are established and defined as significantly higher. Here, BM_{T} is used in conjunction with the BM_{L} for a person to create a delta value (Δ) that is indicative of the disease progression.

The larger the Δ value the larger the disease progression. Each individual will be characterized by his/her own Δ value for the specific BM and will be defined as Δ_{BM}:

Δ_{BM}=BM_{L}−BM_{T} 1)

For example, the standard hs-CRP cut-off point or BM_{T} for high-risk of future development of coronary heart disease or CHD is 3.0 μg/mL. In this case, a person with a CRP BM_{L}=2 μg/mL would be considered having no risk for CHD (Δ_{CRP}=−1), while a person with a CRP BM_{L}=5 μg/mL would have a significantly higher Δ_{CRP}=3 and would be considered having low risk for CHD; another person with CRP BM_{L}=10 μg/mL would be considered as a HIGH RISK individual with significant risk CHD progression (Δ_{CRP}=7).

This method utilizes a multi-biomarker approach to assess risk for developing a disease or to diagnose a disease. Accordingly, multiple values of Δ_{BM} will be generated, one for each BM associated with each disease (Δ_{BM#1}, Δ_{BM#2} . . . Δ_{BM#n}).

Since not all BMs contribute to the same extent to the development of a specific disease, each of the Δ_{BM} will be converted to a weighted Δ_{BM} (ΔDISEASE_{BM}) by incorporating a weighing factor (W_{DISEASE-BM}) that will be based on the published increase in risk associated with the BM at the indicated concentration for the development of the specific disease.

ΔDISEASE_{BM}=Δ_{BM}×W_{DISEASE-BM}=(BM_{L}−BM_{T})×W_{BM} 2)

As another example, for BM CRP with BMT at 3.0 μg/mL and for an individual with BM_{L}=10 μg/mL:

ΔCHD_{CRP}=(10−3)×W_{CHD-CRP}=7×3=21

The method next calls for an accumulated ΔDISEASE value to be derived collectively from all the BMs associated with the specific disease. This ΔDISEASE value will define the risk for an individual to develop (or to have) the specific disease based on multiple biomarkers. As such, this value will be defined as follows:

ΔDISEASE=Σ ΔDISEASE_{BM#1} . . . ΔDISEASE_{BM#}ₙ 3)

n is defined is the total number of biomarkers.

For example, in this embodiment, cardiac risk may be defined by the levels of the following biomarkers:

1. C-reactive protein (CRP)

2. soluble CD40 ligand (sCD40L)

3. monocyte chemoattractant protein-1 (MCP-1)

4. myeloperoxidaxe (MPO)

5. interleukin-1b (IL-1β)

6. IL-6

7. tumor necrosis factor-a (TNF-a)

8. Homocysteine

9. phospholipase A2 (PLA2)

10. Lipoprotein A (LpA)

DCHD=DCHD_{CRP}+DCHD_{CD40L}+DCHD_{MCP-1}+DCHD_{MPO}+DCHD_{IL-1b}+DCHD_{IL-6}+DCHD_{TNF}+DCHD_{homocysteine}+DCHD_{PLA2}+DCHD_{LpA}

Here each biomarker will be weighed differently. An adjustment factor may be implemented for those BMs that represent the same pathophysiological process as well as for those that act in a synergistic fashion for the development of this specific disease.

Therefore, for somebody with more than one BM elevated above BM_{T} as follows:

1. C-reactive protein (CRP): DCHD_{CRP}=21

2. soluble CD40 ligand (sCD40L): DCHD_{CD40L}=7

3. monocyte chemoattractant protein-1(MCP-1): DCHD_{MCP-1}=5

4. myeloperoxidase (MPO): DCHD_{MPO}=4

5. interleukin-1b (IL-1b): DCHD_{IL-1b}=3

6. IL-6: DCHD_{IL-6}=6

7. tumor necrosis factor-a (TNF-a): DCHD_{TNF}=2

8. Homocysteine: DCHD_{homocysteine}=10

9. phospholipase A2 (PLA2):+DCHD_{PLA2}=5

10. Lipoprotein A (LpA): DCHD_{LpA}=4

DCHD=21+7+5+4+3+6+2+10+5+4=59

This method will then incorporate all biomarkers of the disease or risky lifestyle behavior to derive the index for risk for death from that disease (Index (for risk for) Death-ID). This ID will incorporate the BM-based index for developing (or having) a disease ΔDISEASE previously calculated (see above) and a disease-specific weighing factor (DW) that will be derived from the mortality data associated with that specific disease.

ID_{Disease}ΔDISEASE×DW (4)

For example, the ID for CHD would be:

ID_{CHD}=ΔCHD×0.31

| Deaths | % of total population |
|---|---|
| Cancer | 0.19 |
| CHD | 0.31 |
| Diabetes | 0.03 |
| HIV | 0.00 |
| All other | 0.30 |

Where, DW=0.31=% of deaths from total population attributed to CHD.

This way in the example used for CRP alone for the CHD case/BM CRP with BMT at 3.0 μg/mL and for an individual with BM_{L}=10 μg/mL

ID_{CHD}=ΔCHD×0.31

ΔCHD=ΔCHD_{CRP}

ΔCHD_{CRP}=(10−3)×W_{CHD-CRP}=7×3=21

ID_{CHD}=21×0.31=6.51

Therefore, somebody with more than one cardiac risk BM elevated above BTM, the ID_{CHD} will be:

1. C-reactive protein (CRP): DCHD_{CRP}=21

2. soluble CD40 ligand (sCD40L): DCHD_{CD40L}=7

3. monocyte chemoattractant protein-1 (MCP-1): DCHD_{MCP-1}=5

4. myeloperoxidase (MPO): DCHD_{MPO}=4

5. interleukin-1b (IL-1b): DCHD_{IL-1b}=3

6. IL-6: DCHD_{IL-6}=6

7. tumor necrosis factor-a (TNF-a): DCHD_{TNF}=2

8. Homocysteine: DCHD_{homocysteine}=10

9. phospholipase A2 (PLA2):+DCHD_{PLA2}=5

10. Lipoprotein A (LpA): DCHD_{LpA}=4

DCHD=21+7+5+4+3+6+2+10+5+4=59

ID_{CHD}=59×0.31=18.3

Note how the index for risk for death from CHD increased from 6.51 for one biomarker (CRP) to 18.3 for multiple biomarker expression.

As the probability to die increases in the presence of multiple diseases and risk factors, this method will then incorporate all diseases or risky lifestyle behaviors to derive the Index for Total Risk for Death (ID_{Total}). This comprehensive index for death will incorporate the BM-based indices for dying by factoring in all of the diseases that afflict an individual:

ID_{Total}=ID_{Disease#1}+ID_{Disease#2}+ . . . +ID_{Disease#n} 5)

Summing here serves to take into account all diseases that may have some redundant biomarkers but distinct biomarker fingerprints and therefore associated Ws and IDs. As such a person accumulating risk factors or behaviors (i.e., obesity+smoking+drinking+drugs+HIV) will have a larger ID total than someone with just diabetes for example. Therefore, for a cardiac patient that exhibits significant chronic level elevations of more than one cardiac risk BM elevation above BM_{T} that also happens to be a heavy daily smoker with cotinine levels 100× above the reference range, ID_{Total} will be:

ID_{Total}=18.3+ID_{Smoker}−18.3+(100×0.134)=18.3+13.4 ID_{Total}=31.7

Death rates due to cigarette smoking in the US rank among the highest in the world, according to the book Mortality from Smoking in Developed Countries 1950-2000. In 1990, smoking accounted for 400,000* (*400,000/296,844,000=0.134%) deaths nationally, says the Center for Disease Control and Prevention (CDC).

According to the 1995 National Health Interview Survey (NHIS), about 47 million adults were current smokers, either daily (20.1 percent) or on some days (4.6 percent). About 32 million adults (68.2 percent) reported they wanted to quit smoking completely, and about 17.3 million (45.8 percent) had quit for at least one day during the past year.

The ID_{Total} will then be used to derive an index of Wellness and Longevity (“LongWell Index” or LWI) as follows:

LongWell Index=(100−ID_{Total}) expressed in % total life expectancy 6)

Here, with this BM-based method, good habits and no disease would presumably result in lowering of ID_{Total}, in best cases to 0, which translates to 100% life expectancy. In contrast, poor lifestyle and habits as well as presence of disease would increase the ID_{Total} or index of Death, which would lead to a lower LongWell Index.

Therefore, for a cardiac patient that exhibits significant chronic level elevations of more than one cardiac risk BM elevation above BTM, that also happens to be a heavy daily smoker with cotinine levels 100× above the reference range,

ID_{Total}=18.3+ID_{Smoker}=18.3+(100×0.134)=18.3+13.4=31.7

The LongWell Index will be:

LongWell Index=(100−31.7)=68.3% total life expectancy

The methods described here guarantee the highest analyte diversity with sensitivity and specificity superior to traditional methods using one analyte at a time or new methods with point of care service with multi-analyte testing capacity.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

The Longevity/Wellness Index (LWI) expressed in % total life expectancy is determined according to equation 1):

LWI=(100−ID_{Total}) 1)

wherein ID_{Total} is the overall total index of death and is determined by equation 2):

ID_{Total}=ID_{Disease#1}+ID_{Disease#2}+ . . . +ID_{Disease#n} 2)

wherein n is the number of diseases considered, and wherein ID_{Disease} is index for risk for death from that disease is determined by equation 3):

ID_{Disease}=ΔDISEASE×DW 3)

wherein ΔDISEASE=Σ ΔDISEASE_{BM#1} . . . ΔDISEASE_{BM#}ₙ

wherein DW is disease-specific weighing factor and ΔDISEASE_{BM} is determined by equation 4:

ΔDISEASE_{BM}=(BM_{L}−BM_{T})×W_{DISEASE-BM} 4)

wherein BM_{L} is the biomarker level in the patients and BM_{T} is the biomarker threshold level which is cut-off point established for the specific marker above or below which disease occurs,

and wherein W_{DISEASE-BM} is a weighing factor based on the published increase in risk associated with a particular level of biomarker for a particular disease.

References:

The following references, to the extent that they provide exemplary procedural or other details supplementary to those set forth herein, are specifically incorporated herein by reference.

US2008300798, US2008038738, US2002138208, US2002193950, US2003004402, US2003055615, WO2000004372, WO2001031580, WO2001006239, WO2001006244, WO2001006253, WO2001055701, WO2001055702, WO2001055703, WO2001055704, WO2001055952, WO2002061392, WO2003090605, WO2004009840, WO2004072097, WO2004072613, WO2005059551, WO2005083423, WO2005085796, WO2005085854, WO2005085855, WO2005090983, WO2007002480, WO2007005666, WO2007134189, WO2007134191

Christodoulides N, et al., A microchip-based multianalyte assay system for the assessment of cardiac risk, Anal. Chem. 74(13):3030-6 (2002).

Christodoulides et al., Application of microchip assay system for the measurement of C-reactive protein in human saliva, Lab. Chip, 5(3):261-9 (2005).

Floriano P N, et al., Use of saliva-based nano-biochip tests for acute myocardial infarction at the point of care: a feasibility study. Clin Chem. 55(8):1530-8 (2009).

Goodey et al., Development of multianalyte sensor arrays composed of chemically derivatized polymeric microspheres localized in micromachined cavities, J. Amer. Chem. Soc., 123(11):2559-2570 (2001).

Jokerst J V, et al., Nano-bio-chips for high performance multiplexed protein detection: determinations of cancer biomarkers in serum and saliva using quantum dot bioconjugate labels, Biosens Bioelectron 24(12):3622-9 (2009).

Gustaysson, P. E., et al. Superporous agarose beads as a hydrophobic interaction chromatography support, J. of Chromatog., A 830(2): 275-284 (1999).

Gustaysson, P. E. and Larsson, P. O., Superporous agarose, a new material for chromatography, J. Chromatog. A 734(2): 231-240. (1996).

We claim:

1. A method for assessing the health and wellness status of a subject comprising the steps of:
a) collecting a sample from a subject;
b) measuring a plurality of biomarkers in said sample to generate a plurality of biomarker levels;
c) determining a wellness index of the subject based said biomarker levels according to the following:
a Longevity/Wellness Index (LWI) expressed in % total life expectancy is determined according to equation 1):
LWI=(100−ID^{Total)} 1)
wherein ID^{Total} is the overall total index of death and is determined by equation 2):
ID_{Total}=ID_{Disease#1}+ID_{Disease#2}+ . . . +ID_{Disease#n} 2)
wherein n is the number of diseases considered and ID_{Disease} is the index for risk for death from that disease is determined by equation 3):
ID_{Disease}=ΔDISEASE×DW 3)
wherein ΔDISEASE is Σ ΔDISEASE_{BM#1} . . . ΔDISEASE_{BM#n}
wherein m is the number of biomarkers considered, and DW is a disease-specific weighing factor and ΔDISEASE_{BM} is determined by equation 4:
ΔDISEASE_{BM}=(BM_{L}−BM_{T})×W_{DISEASE-BM} 4)
wherein BM_{L} is the biomarker level in the patients and BM_{T} is the biomarker threshold level which is cut-off point established for the specific marker above or below which disease occurs, and wherein W_{DISEASE-BM} is a weighing factor based on the published increase in risk associated with a particular level of biomarker, and
d) reporting said wellness index to said subject or said subject's medical professional.

2. The method of claim 1, wherein the biomarkers are cardiovascular disease biomarkers, cancer biomarkers, diabetes biomarkers, and inflammation biomarkers, and optionally stress biomarkers, smoking biomarkers, drug-abuse biomarkers, infectious disease biomarkers, age, gender, ethnicity, overweightness, high abdominal fat, or combinations thereof.

3. The method of claim 2, wherein the biomarkers are selected from the markers in Table 1.

4. The method of claim 1, wherein the sample is saliva.

5. The method of claim 1, wherein the measuring is conducted on a microarray device.

6. The method of claim 1, wherein the measuring is conducted on a microarray device wherein said microarray is comprised of an array of agarose disks on a porous substrate.

7. (canceled)

8. A microarray comprising a porous substrate onto which an array of testing sites are located, each testing site comprising a flat agarose pad, the agarose further comprising one member of a target-detector binding pair conjugated thereto.

9. The microarray of claim 8, wherein a length and width of said flat agarose pad is at least 10 times greater than a height of said flat agarose pad.

10. The microarray of claim 8, wherein said member of a target-detector binding pair is one or more drugs or drug antibody.

11. The microarray of claim 10, wherein said drugs are selected from the group consisting of Δ9-tetrahydrocannabinol (THC), cocaine, amphetamine and diazepam.

12. The microarray of claim 10, wherein the microarray is a disposable cassette that operably fits into a portable device that provides power, fluid pumping, control and display functions.

13. The microarray of claim 8, wherein the microarray further comprises microfluidics to connect said flat agarose pad to a regent reservoir and a waste reservoir.

14. The microarray of claim 8, wherein the microarray further comprises microfluidics to connect said flat agarose pad to a regent reservoir above said flat agarose pad and a waste reservoir below said flat agarose pad.

15. A portable drug testing device, wherein said diagnostic comprises an array of flat agarose pads on a porous substrate,
wherein said array includes:
i) multiple flat agarose test pads wherein said agarose is conjugated to a drug or drug antibody,
ii) multiple negative control flat agarose pads, and
iii) multiple positive control flat agarose pads wherein said agarose is conjugated to a known amount of said drug or drug antibody,
wherein said array operably fits into a portable device that provides power, fluid pumping, and control and display functions.

16. The portable drug testing device of claim 15, wherein said drugs are selected from the group consisting of Δ9-tetrahydrocannabinol (THC), cocaine, amphetamine and diazepam and said agarose is conjugated to antibodies to said drugs, such that a competitive immunoassay can be performed on said device.

17. The portable drug testing device of claim 15, wherein said array further comprises fluidics to connect said flat agarose pads to a regent reservoir and a waste reservoir.

18. The portable drug testing device of claim 15, wherein the array further comprises fluidics to connect said flat agarose pad to a regent reservoir above said flat agarose pad and a waste reservoir below said flat agarose pad.
